(19) United States
(12) Patent Application Publication
(10) Pub. No.: US 2018/0296695 A1
(43) Pub. Date: Oct. 18, 2018

(54) hnRNPA2B1-FAM96A fusion protein and composition for diagnosing cancer

(71) Applicant: Ministry of Food and Drug Safety, Cheongju-si (KR)

(72) Inventors: Ze Won PARK, Sejong-si (KR); Dae Young ZANG, Seoul (KR); Young Ho KOH, Anyang-si (KR)

(21) Appl. No.: 15/949,070

(22) Filed: Apr. 10, 2018

(30) Foreign Application Priority Data
Apr. 14, 2017 (KR) 10-2017-0048417

(51) Int. Cl. A61K 47/68; A61K 31/7088; G01N 33/574; A61K 47/64

(52) U.S. Cl. CPC A61K 47/6897; A61K 31/7088; A61P 35/00; A61K 47/6425; A61K 47/6851; G01N 33/57484

(57) ABSTRACT

Background: Recently, studies on genome and transcritome of gastric cancer have suggested that gastric cancer is a heterogeneous disease caused by various genetic defects combined with environmental risk factors. In the present invention, a fusion protein expressed only in Korean gastric cancer tissues is detected by performing quantitative label-free proteome analysis.

Result: A heterogeneous nuclear ribonucleoprotein A2/B1 (hnRNPA2B1)-family with sequence similarity 96 member A (FAM96A) fusion protein, which is not expressed in a normal gastric tissue, but expressed only in a gastric cancer tissue, is identified.

### CROSS-REFERENCE TO RELATED APPLICATIONS

This application claims the priority of the Korean Patent Application NO 10-2017-0048417 filed on Apr. 14, 2017 in the Korean Intellectual Property Office, the disclosure of which is incorporated herein by reference.

### SEQUENCE LISTING

A SEQUENCE LISTING is submitted in a file named pus180022_5 T25 via EFS Web and is hereby incorporated by reference in its entirety. Said file was created on Apr. 6, 2018 and is 2,360 bytes in size.

### TECHNICAL FIELD

The present invention relates to an hnRNPA2B1-FAM96A fusion protein and a composition for diagnosing cancers including the same, and more particularly, to an hnRNPA2B1-FAM96A fusion protein and a composition for diagnosing cancers including the same, which is not expressed in a normal gastric tissue, but expressed only in a gastric cancer tissue.

### BACKGROUND ART

Gastric cancer is the third most common cause of tumor-related death worldwide. Further, the gastric cancer is the next most common tumor to thyroid cancer in 2013. The genetic basis of the gastric cancer has been extensively studied in recent years, and the gastric cancer is not a homogenous disease, but a complex of diseases caused by a combination of many genetic mutations and environmental risk factors. Several genetic studies on the gastric cancer have suggested that mutations identified in gastric cancer tissue vary according to a gastric cancer type, age of onset, race, and gastric cancer sites. Numerous mutations, including both germinal mutation and somatic mutation, appear differently in individual gastric cancer tissues. In contrast to genetic studies, proteome studies on the gastric cancer have not been widely performed until now, although signaling pathways are mostly regulated by proteins expressed in cells and tissues.

One of major objectives of the tumor proteome study is to check diagnostic biomarkers for tumor patients by identifying proteins expressed differently in the tumor tissues. Compared to other tumors, gastric cancer diagnostic and prognostic biomarkers have not been extensively defined up to this day. A clinically applicable diagnostic marker for the gastric cancer is human epidermal growth factor receptor 2 (HER2), which is first identified in breast cancer and observed in about 20% of patients with recurrent or metastatic tumors.

Among mutations that cause tumors, fusion genes derived from chromosome transfer and rearrangement are well known as driver mutations that play an important role in many types of tumors. The first and best studied fusion gene has been identified in chronic myelogenous leukemia (CML) and has occurred through the fusion of a breakpoint cluster region (BCR) gene and an abelson murine leukemia viral oncogene homolog 1 (ABL1) gene. The identification of the BCR-ABL1 fusion gene in the CML has induced the development of a targeted anticancer agent imatinib which is a tyrosine kinase inhibitor. The fusion genes are also found even in solid cancers. For example, it has been reported that the fusion of an echinoderm microtubule-associated protein-like 4 (EML4) gene and an anaplastic lymphoma receptor tyrosine kinase (ALK) gene occurs in 4% of non-small-cell lung cancer patients. EML4-ALK-positive patients showed a significant clinical effect when prescribed with an ALK inhibitor crizotinib (PF02341066).

Recent studies on the effect of cancer prescription therapy have suggested that a genetic difference between cancer patients may be the most important factor to determine the therapeutic effect. Thus, a recent trend in cancer patient therapy is to identify genetic defects in cancer patients and then prescribe a specific target drug to this mutation. One example of the targeted gastric cancer drugs is herceptin, which is a monoclonal antibody that specifically binds to the HER2 to inhibit the activity. The herceptin may have a good clinical effect when prescribed to about 20% of the HER2-positive gastric cancer patients in combination with a chemical therapy. Other targets for the targeted gastric cancer therapy under development include a vascular endothelial growth factor receptor 2, a human epidermal growth factor receptor 3, a phosphatidylinositol 4,5-diphosphate 3-kinase catalytic subunit-α, an MET and a fibroblast growth factor receptor.

For completely individualized medicine, other genetic defects in gastric cancer need to be found. In the present invention, a quantitative and qualitative label-free proteomic analysis is used to identify proteins (DEPs) and fusion proteins having different expressions that are not present in normal tissues, but are present only in gastric cancer tissues. The identified DEPs and fusion genes may be used as cancer diagnostic and prognostic markers and targets of tools for developing precise medicine. Accordingly, in order to develop novel druggable targets and target therapies for the gastric cancer, extensive genetic studies are required.

### PRIOR ART DOCUMENT

### Non-Patent Documents

1. Ferlay J, Soerjomataram I, Ervik M, Dikshit R, Eser S, Mathers C, Rebelo M, Parkin D M, Forman D, Bray F: v1.0, Cancer Incidence and Mortality Worldwide: sources, methods and major patterns in GLOBOCAN 2012, International Journal of Cancer; vol. 136, 2015.
2. TCGA: Comprehensive molecular characterization of gastric adenocarcinoma. Nature 2014, 513(7517):202-209.

The mention of any reference in the present application does not permit that the references are prior arts related to the present application.

### DISCLOSURE

### Technical Problem

An object of the present invention is to provide more accurate and simple diagnostic and prognostic markers for gastric cancer.

### Technical Solution

The development of a new therapy method for gastric cancer has contributed to a significant reduction in mortality worldwide. Nevertheless, the gastric cancer is still one of the most common cancers in Korea and Asia. When considering the fact that the gastric cancer is associated with heterologous gene defects, specific genetic defects at a protein level of each patient need to be found to extend the efficacy of the individualized medicines.

In the present invention, a label-free proteome analysis was performed in order to study proteins (DEPs) and fusion proteins of which expression is changed in Korean gastric cancer tissues. The DEPs identified in the present invention may be divided into six closely related clusters and five relatively associated groups. These five groups are manually summarized on the basis of reported or predicted functions, since the main reason of the direct interaction among the groups is not yet clear (FIG. 2). The six clusters identified in the present invention are targeted not only to proteins known to play a crucial role in carcinogenesis and metastasis, but also to recently prescribed cancer therapeutic agents or analogs thereof. For example, various β-tubulins are known targets of Paclitaxel and Vinca alkaloids that inhibit chromosomal mitosis and angiogenesis in tumor cells, and these targets are clustered (FIG. 2II). In addition, a heat shock molecule chaperone, which is increased in most cancers, is significantly increased and clustered even in gastric cancer (FIG. 2V). In addition, factors, which are present in the endoplasmic reticulum and the Golgi body and regulate folding, modification, and trafficking of proteins, are also significantly increased and clustered. Among these proteins, RPN2, a component of an N-oligosaccharide transferase complex associated with resistance to anticancer agents in breast cancer and non-small cell lung cancer, is significantly increased to indicate the possibility of preserving drug resistance and metastatic mechanisms in gastric cancer. In addition, a significant increase in HSPA5, CALR and CANX in gastric cancer activates an unfolded protein response (UPR) to overcome hypoxia and low nutrition supply in gastric cancer, and this is similar to UPR activation observed in helicobacter-induced gastric cancer, and the hypoxia and the low nutrition supply are two initial stress conditions observed in tumor cells and induce aggression in a melanoma gastric model. Interestingly, the present inventors have identified clusters of significantly up-regulated glycolytic enzymes, such as PKM, PGK1, ENO1, ENO2 and GPI (FIG. 2IV). Recent studies on tumor metabolism suggest that the up-regulation of the glycolytic enzymes may be associated with carcinogenesis and drug resistance and may be used as predictive biomarkers. When summarizing these findings, it is known that most of the DEPs identified in the present invention are already associated with carcinogenesis, metastasis and drug resistance.

In the present invention, FIG. 3 shows hnRNAPA2B1 fusion proteins identified in gastric cancer. The use of targeted anticancer agents has several advantages such as fewer side effects and a higher response rate. However, only one type of targeted anti-gastric cancer agent, herceptin, has been currently used for the gastric cancer therapy, and the herceptin is effective in only about 20% of the HER2-positive gastric cancer patients. Therefore, new target genes or proteins need to be found in order to realize precise medicine in gastric cancer. The hnRNAPA2B1 fusion protein identified in the present invention is very interesting when considering that the ALK fusion gene is a driver mutation that plays an important role in several tumors and is a target of development of the targeted anticancer agent. The hnRNAPA2B1 regulates expression of tumor suppression genes and down-regulation of FAM96A reported in the gastrointestinal stromal tumor (GISTs). Furthermore, a three-dimensional homology modeling of the fusion proteins suggests that the fusion proteins may form heterodimers or homodimers with normal proteins or fusion proteins. In the dimerization result, the fusion proteins were found at a place where normal proteins were not located, resulting in abnormal activity of hnRNPA2B1 or FAM96A. The abnormal activity of such a hnRNAPA2B1 fusion protein may change normal cells into cancer cells.

The present invention provides a hnRNPA2B1-FAM96A fusion protein in which a heterogeneous nuclear ribonucleoprotein A2/B1 (hnRNPA2B1) protein or a fragment thereof at an N-terminal and a family with sequence similarity 96 member A (FAM96A) protein or a fragment thereof at a C-terminal are fused. Further, the present invention is characterized in that the hnRNPA2B1-FAM96A fusion protein has an amino acid sequence of SEQ ID NO: 1. Additionally, the present invention provides a hnRNPA2B1-FAM96A fusion gene encoding the fusion protein. Further, the present invention is characterized in that the fusion gene has a nucleotide sequence of SEQ ID NO: 2.

Further, the present invention provides a composition for diagnosing cancers including at least one selected from the group consisting of a molecule specifically binding to the hnRNPA2B1-FAM96A fusion protein and a molecule specifically binding to the fusion gene encoding the fusion protein. In addition, the present invention is characterized in that the molecule specifically binding to the fusion protein is selected from the group consisting of an antibody and an aptamer. Further, the present invention is characterized in that the cancer is gastric cancer. Further, the present invention relates to a method for providing cancer diagnosis information including detecting the fusion protein, a fusion gene encoding the fusion protein, or an mRNA corresponding to the fusion gene in a biological sample isolated from a patient to be tested, in which when the fusion protein, the fusion gene, or the mRNA is detected, the patient is determined as a cancer patient.

Further, the present invention relates to a composition for preventing or treating cancers including at least one selected from the group consisting of an inhibitor of the fusion protein and a polynucleotide molecule inhibitor encoding the fusion protein as an active ingredient.

For example, in the method for providing the cancer diagnosis information, the cancer is gastric cancer. In one example, the fusion protein and/or the fusion gene according to the present invention has been identified to be specifically found or expressed in patients with solid cancer, specifically, gastric cancer, and thus, the fusion protein and/or the fusion gene encoding the fusion protein is useful as a diagnostic marker for the solid cancer, specifically, the gastric cancer.

The present invention provides a composition for diagnosing cancers including a molecule specifically binding to the fusion protein and/or a polynucleotide hybridizable with the fusion gene. The molecule specifically binding to the fusion protein may be selected from the group consisting of an antibody, an aptamer, and the like. In addition, the polynucleotide hybridizable with the fusion gene encoding the fusion protein refers to a polynucleotide which has 20 to 100, specifically 25 to 50 base sequences adjacent to both terminals of a DNA molecule, or complementary base sequences which are completely complementary, or 80% or more, preferably 90% or more complementary with the base sequences so as to amplify the DNA molecule consisting of 50 to 250, specifically 100 to 200 consecutive bases including consecutive fusion sites in the fusion gene.

The present invention provides a method for providing cancer diagnostic information including measuring the expression of the fusion protein in a biological sample obtained from a patient. In the method, when the expression of the fusion protein is detected in the biological sample, the patient may be determined as a patient with cancer (solid cancer), specifically, gastric cancer. The detecting of the expression of the fusion protein may be performed by detecting the presence of the fusion protein in the biological sample or detecting the presence of the fusion gene encoding the fusion protein or the corresponding mRNA. For example, the presence of the fusion protein may be detected by a general analysis method for detecting the interaction of the fusion protein with the molecule (e.g., an antibody or aptamer) using the molecule (e.g., an antibody or aptamer) specifically binding to the fusion protein, such as immunochromatography, immunohistochemical staining, enzyme-linked immunosorbent assay (ELISA), radioimmunoassay (MA), enzyme immunoassay (EIA), fluorescence immunoassay (FIA), luminescence immunoassay (LIA), western blotting, FACS, and the like.

An immunoassay may be performed by a general protein expression detection assay. A useful immunoassay may be a homogeneous immunoassay or a heterologous immunoassay. In the homogeneous assay, the immunological response is often associated with a fusion protein-specific reagent, such as a fusion polypeptide-specific antibody, a labeled analyte, and a biological sample to be analyzed. A signal generated by the label is directly or indirectly modified by binding of the antibody to the labeled analyte. Usable immunochemical labels may be at least one selected from the group consisting of free radicals, radioisotopes, fluorescent dyes, enzymes, bacteriophages, coenzymes, and the like.

Antibodies useful in the performance of the methods described in the specification, claims, and the like may be attached to solid supports (e.g., wells, beads, plates or slides made of materials such as latex or polystyrene) suitable for diagnostic analysis. Antibodies or other fusion protein binding reagents may be also attached to detectable functional groups such as radioactive labels (e.g., 35S, 125I, 131I, etc.), enzyme labels (e.g., horseradish peroxidase, alkaline phosphatase, etc.), and fluorescent labels.

The patients may be mammals including primates such as humans and monkeys, and rodents such as mice and rats. The biological sample may be cells, tissues, body fluids, etc. isolated from the patients.

The fusion protein specifically expressed in the cancer patient of the present invention may be used as a new cancer therapeutic target. Therefore, another exemplary embodiment of the present invention provides a composition for preventing and/or treating cancers including at least one selected from the group consisting of an inhibitor of the fusion protein and a polynucleotide molecule inhibitor encoding the fusion protein as an active ingredient. The inhibitor of the fusion protein is a substance which binds to the fusion protein and loses or lowers its function and may be at least one selected from the group consisting of an antibody against the fusion protein, an aptamer, or a general kinase inhibitor, a signal transduction inhibitor, and the like. The fusion DNA molecule inhibitor encoding the fusion protein is a substance that binds to the DNA molecule so as not to be expressed to the fusion protein and may be at least one selected from the group consisting of an siRNA, an shRNA, an aptamer, and the like which specifically bind to the DNA molecule.

Cancers to be diagnosed or treated by the composition for diagnosing cancers, the method for providing the cancer diagnostic information, and the composition for preventing and/or treating the cancers may be all types of solid cancers. For example, the solid cancers may be lung cancer, liver cancer, colon cancer, pancreatic cancer, gastric cancer, breast cancer, ovarian cancer, kidney cancer, thyroid cancer, esophageal cancer, prostate cancer, brain cancer and the like, and particularly, the solid cancer may be gastric cancer.

### Advantageous Effects

According to the present invention, the fusion proteins expressed in the Korean gastric cancer are identified and the identified heterogeneous nuclear ribonucleoprotein A2/B1 (hnRNPA2B1)-family with sequence similarity 96 member A (FAM96A) fusion protein may be used as a gastric cancer marker.

### DESCRIPTION OF DRAWINGS

FIG. 1 shows a result of analyzing gene ontology, that is, up-regulated or down-regulated genes in gastric cancer. Up-regulated proteins A or down-regulated proteins B identified in gastric cancer are associated with a variety of molecular functions and biological processes.

FIG. 2 shows a result of search tool for recurring instances of neighboring genes (STRING) analysis of up-regulated (red) or down-regulated (blue) proteins identified in gastric cancer tissues as compared with normal gastric tissues. The up-regulated or down-regulated genes are classified according to main functional characteristics thereof. Six closely related clusters are identified. Depending on the reported or predicted biological and molecular functions, five additional groups are further classified.

FIG. 3 shows a predicted three-dimensional structure of the hnRNPA2B1-FAM96A fusion protein, in which a three-dimensional homology modeling of the hnRNPA2B1-FAM96A fusion protein shows possible dimerization of the fusion protein through the FAM96A dimerization.

### MODES OF THE INVENTION

Hereinafter, configurations of the present invention will be described in more detail with reference to detailed Examples. However, it is apparent to those skilled in the art that the scope of the present invention is not limited to only the disclosure of Examples.

<Materials and Methods>

Clinical Tissue Samples

A total of 9 pairs of gastric cancer tissues and adjacent normal tissues were collected from patients with gastric cancer who provided information and agreed on the gastrectomy received in the Hallym University Sungsim Hospital in 2011. Detailed patient clinical data were summarized in Table 1. As determined by pathologists, a central region of the tumor avoiding a necrotic tissue and adjacent normal tissues were used in Examples of the present invention. This study was approved by the Clinical Trials Committee of the Hallym University Sungsim Hospital (Approval No.: 2011-1057).

Protein Extraction

Proteins were extracted from gastric cancer tissues and adjacent normal tissues for label-free proteomic analysis using a T-PER tissue protein extraction kit (Pierce Biotechnology, Rockford, Ill., USA). Briefly, 20 to 30 mg of the tissue was grinded together with glass beads in 200 μL of a T-PER reagent (containing protease) (Roche Diagnostics, Basel, Switzerland) and then sonicated six times for 30 seconds. The protein extract was centrifuged at 15,000 g for 10 minutes to obtain water-soluble fractions. All steps were performed on ice. The total protein in the water soluble fractions was quantified using BCA analysis. A supernatant was mixed with a loading buffer (Tris 40 mM pH 7.5, 2% SDS, 10% glycerol, and 25 mM DTT) and the mixture was heated at 95° C. for 5 minutes, and then 30 μg of the proteins were loaded for each lane of a 12% SDS-PAGE gel. Thereafter, the gel was cut to separate respective sample lanes, and each lane was divided into five pieces. Finally, each piece was treated with trypsin-gold and then peptides were extracted and completely dried.

Label-Free Quantitative Proteomics

The peptides obtained by trypsin treatment were analyzed three times using a nanoAcquity UPLC (Waters, Milford, Mass.) coupled to a Synapt G1 HDMS mass spectrometer (Waters). The peptides were isolated using a BEH 130 C18 75 μm×250 mm column (Waters) with a particle size of 1.7 μm and concentrated with a Symmetry C18 RP (180 μm×20 mm, particle size of 5 μm). In each experiment, 2 μL of the trypsin-treated peptides were loaded onto a concentration column with a mobile phase A (water containing 0.1% formic acid), respectively. The step concentration gradient was applied at a rate of 280 nL/min, contained 5 to 45% of a mobile phase B (acetonitrile containing 0.1% formic acid) for 55 minutes, and thereafter, the concentration steeply increased to 90% of the mobile phase B for 10 minutes. The eluted peptides were analyzed in a positive ionization mode using a data-independent MSE mode. MS/MS peaks of [Glu1]-fibrinopeptide (400 fmol/μL) were applied for calculating a time-of-flight analyzer (TOF) in a range of m/z 50 to 1990, and double charge [Glu1]-fibrinopeptide ions (m/z 785.8426) were applied for lock mass correction. While the data was obtained, a capillary voltage was set to 3.2 kV and a power supply temperature was set to 100° C. The disintegration energy in a low energy MS mode (complete peptide ions) and a rising energy mode was set to 6 eV and 15 to 40 eV, respectively. A scan time was set to 1.0 second.

A LC-MSE raw data file was processed, and protein identification and relative quantitative analysis were performed using a ProteinLynx Global Server (PLGS 2.5.1, Waters).

Processing parameters included auto-resistance to precursors and resultant ions, at least three fragment ion matches per peptide, at least seven fragment ion matches per protein, at least two peptide matches per protein with up to 4% false positive rate (FRP), carbamidomethylation (+57 Da) of cysteine as a fixed variation, oxidation (+16 Da) of methionine as various variations, and one allowed missed cleavage. The proteins were identified by searching the Homo sapiens database (70,718 entries) on the Unitprot website (http://www.uniprot.org) as a PLGS software ion counting algorithm.

Quantitative analysis was performed using Waters expression, which was a part of PLGS 2.5.1, based on the measurement of peptide ion peak intensities measured and observed repetitively three times in a low collision energy mode. A data set was standardized using an automatic standardization function. All proteins were identified with reliability of >95%, and in three repeated experiments for each sample, the same peptides were clustered using clustering software included in PLGS 2.5.1 based on mass accuracy and a retention time tolerance of <0.25 min. Only proteins identified by repeating at least ⅔ technology device with a protein probability score of 80 or higher were selected for quantitative and qualitative analysis. In order to identify fusion proteins in gastric cancer, the present inventors used a fusion protein database from the Catalog of Somatic Mutations in Cancer (COSMICv77: http://cancer.sanger.ac.uk/cosmic). Amino acid sequence portions of the fusion proteins matched with the peptides were colored as follows.

Matched with one peptide: blue,

Matched with some of peptides: red,

Matched with modified peptides: green, and

Matched with partially modified peptides: yellow.

26 fusion protein candidates were identified according to a manual and whether the peptide binds a junction of two proteins was tested.

Prediction of Three-Dimensional Structure of Fusion Protein

A three-dimensional similarity model of the hnRNPA2B1-FAM96A fusion protein was generated using the MArkovian TRAnsition of Structure evolution: Protein 3-D Structure Comparison (http://strcomp.protein.osaka-u.ac.jp/matras/). Swiss PDB viewer 4.0.1 (Swiss Institute of Bioinformatics) was used for visualization and modification of the 3D fusion protein model.

Western Blot Analysis

Protein extracts derived from normal tissues and cancer tissues were isolated through 12% SDS-PAGE and then transferred to a nitrocellulose membrane. The protein extracts were blocked with a TBST containing 5% defatted milk powder and then proteins were detected from five pairs of gastric cancer samples and a normal control using mouse anti-TPM3 and anti-TPM4 antibodies (Developmental Study Hybridism Bank, University of Iowa, Ames, Iowa, USA). A monoclonal anti-β-actin antibody (Sigma-Aldrich, St. Louis, Mo., USA) was used as a loading control.

Proteins Differentially Expressed in Korean Gastric Cancer Patients

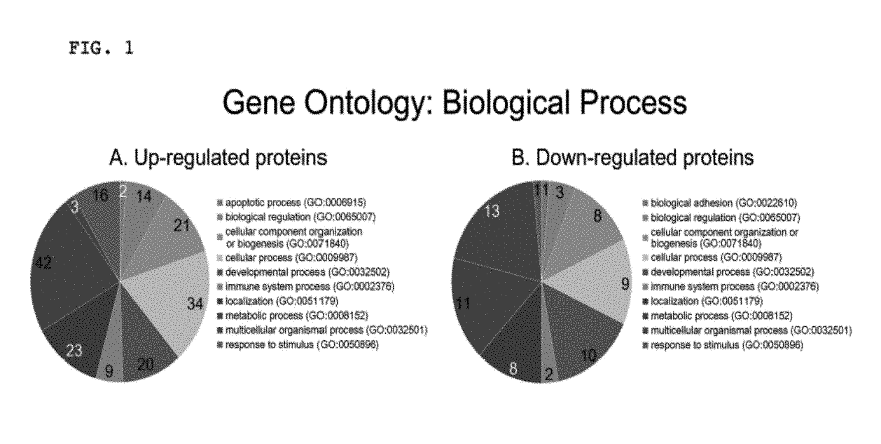

To identify proteins expressed differently in gastric cancer tissues, label-free proteomics was performed using nine pairs of cancers and normal gastric tissues matched thereto. The present inventors found 72 up-regulated proteins or 29 down-regulated proteins in at least five gastric cancer tissues, as compared with a normal control tissue, respectively (FIG. 1).

To study a gene ontology category of the differentially expressed proteins, up-regulated proteins or down-regulated proteins were uploaded in the Panther database (www.pantherdb.org) to be categorized according to biological processes. Among the 72 up-regulated proteins in gastric cancer tissues, 42, 34, 23 and 21 proteins were assigned to categories of a metabolic process, a cellular process, a location and a cellular component organization or biosynthesis, respectively. In contrast, among the 29 down-regulated proteins, 13, 11, 10 and 9 proteins were assigned to categories of a multicellular tissue process, a metabolic process, a developmental process and a cell process, respectively (FIG. 1).

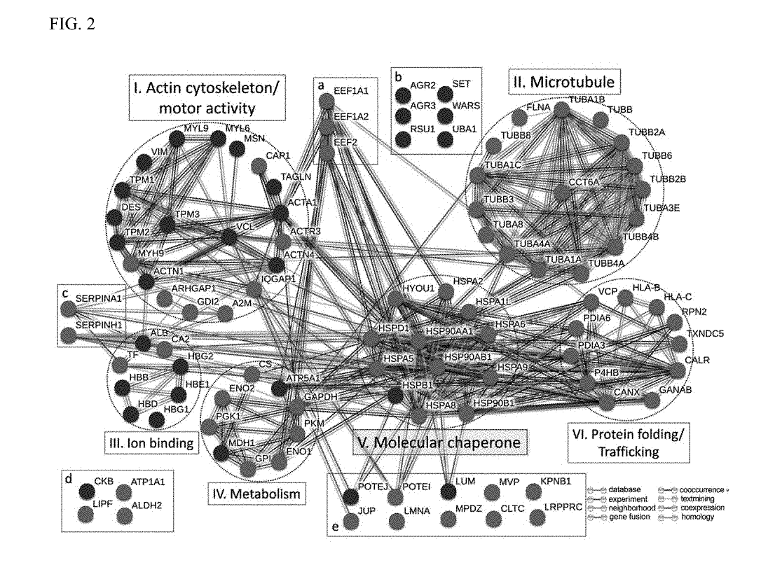

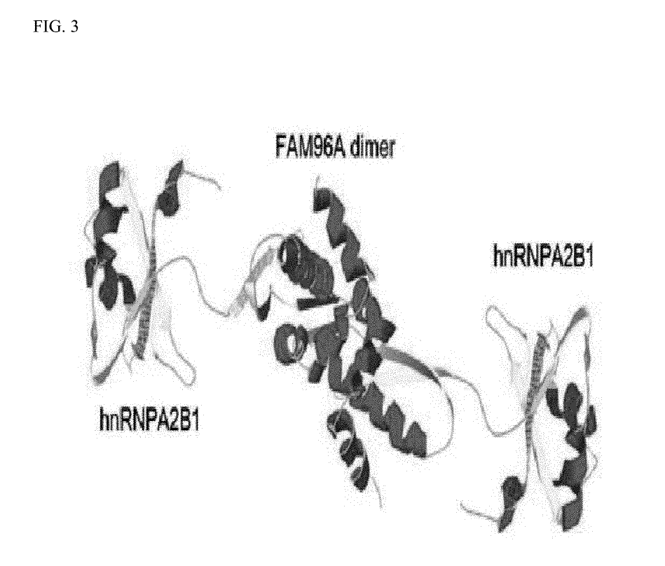

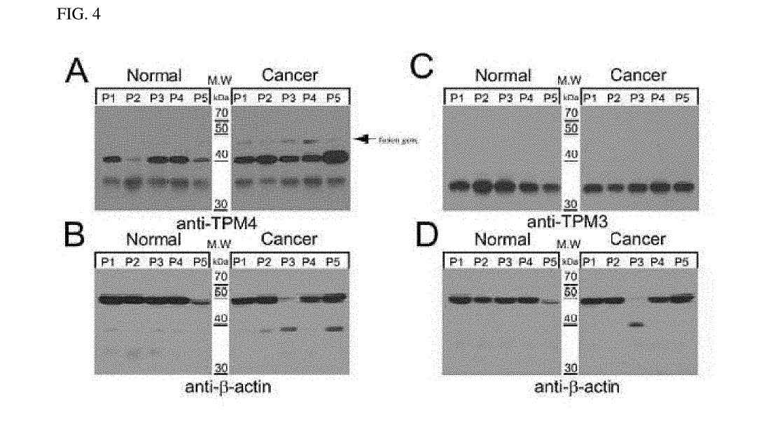

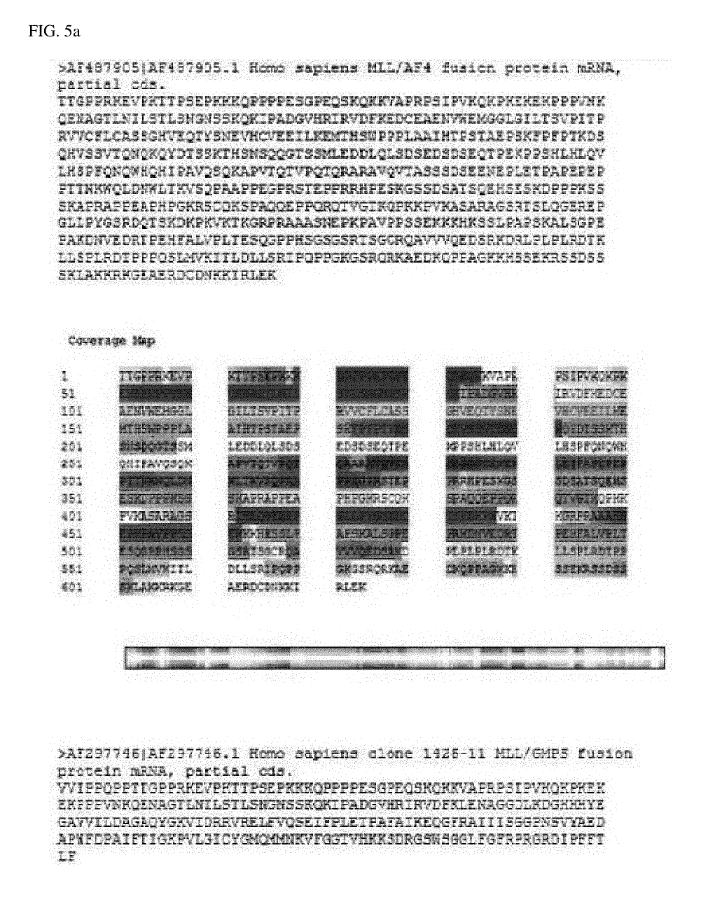

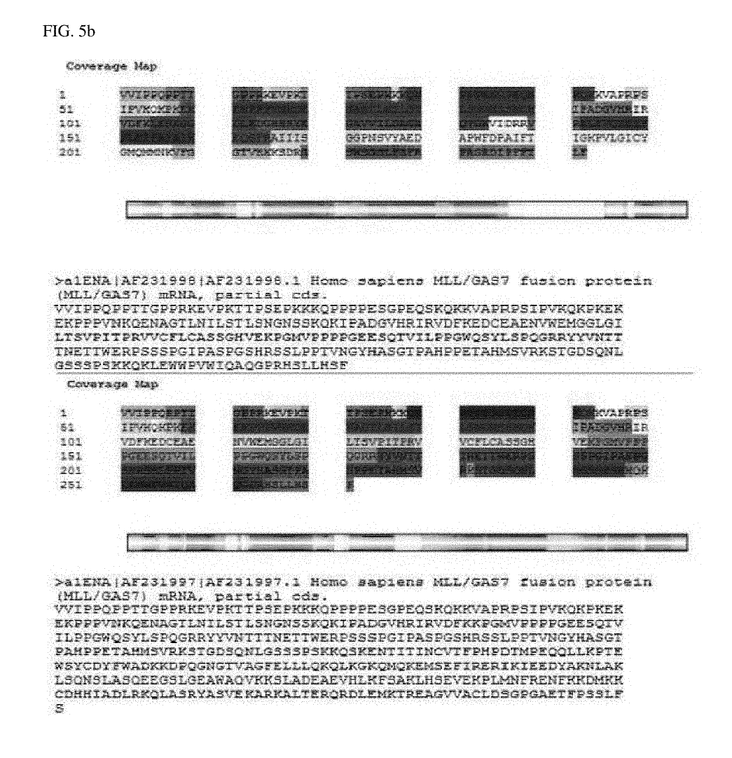

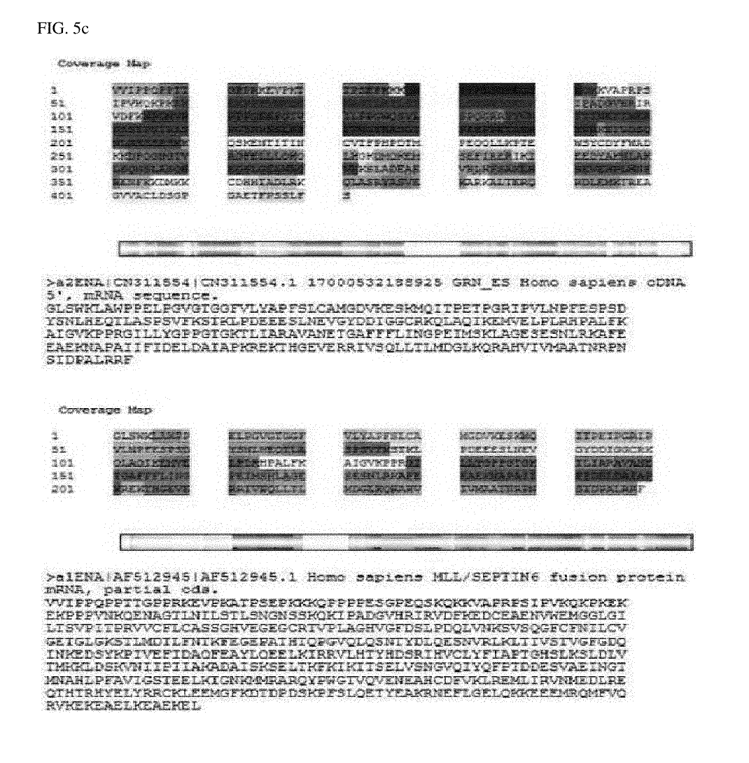

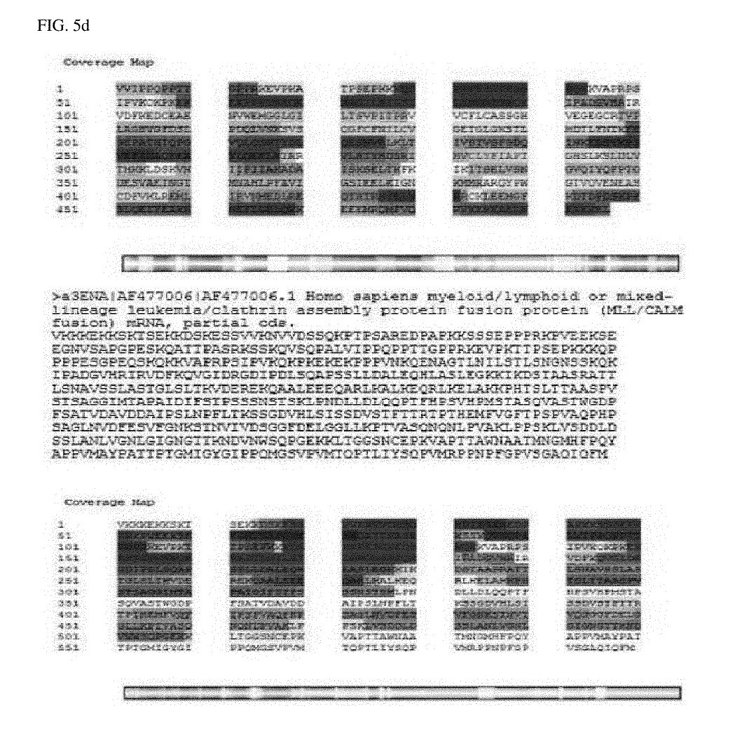

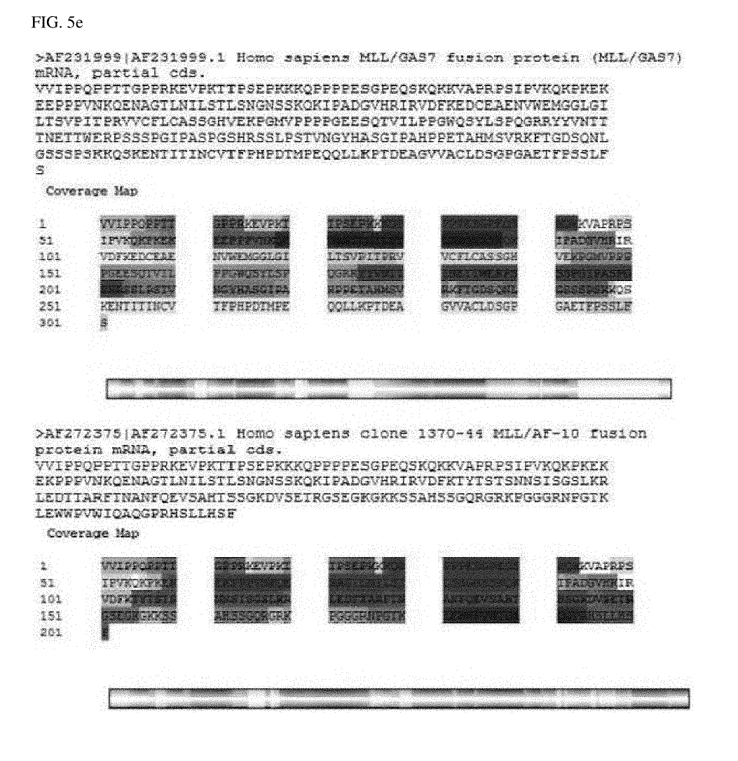

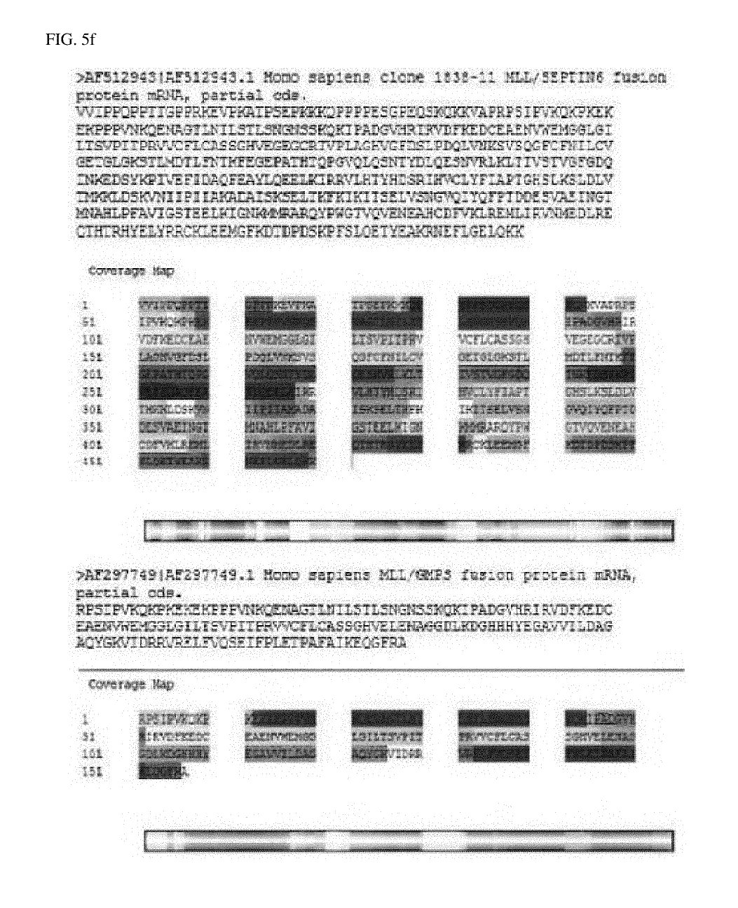

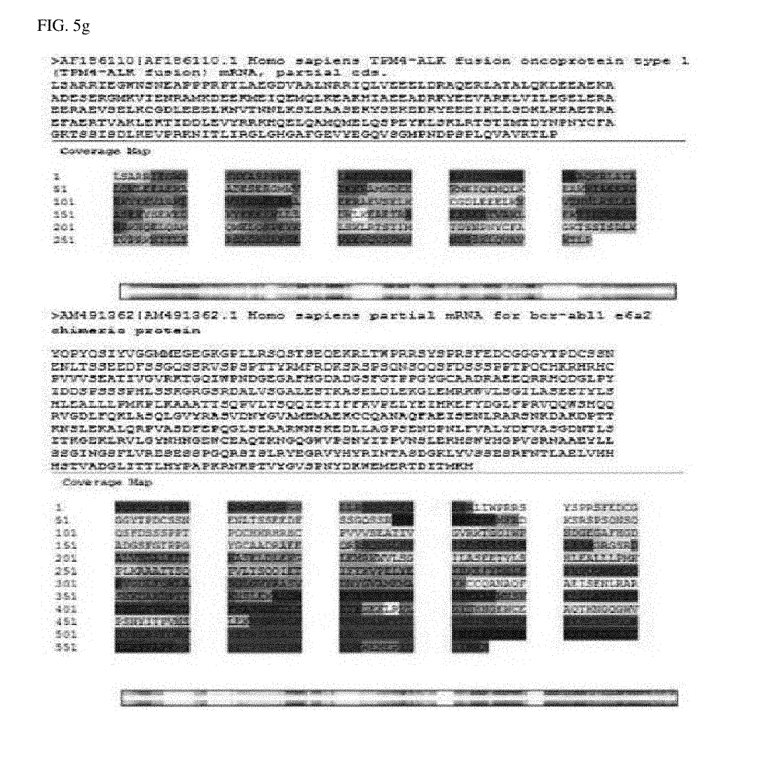

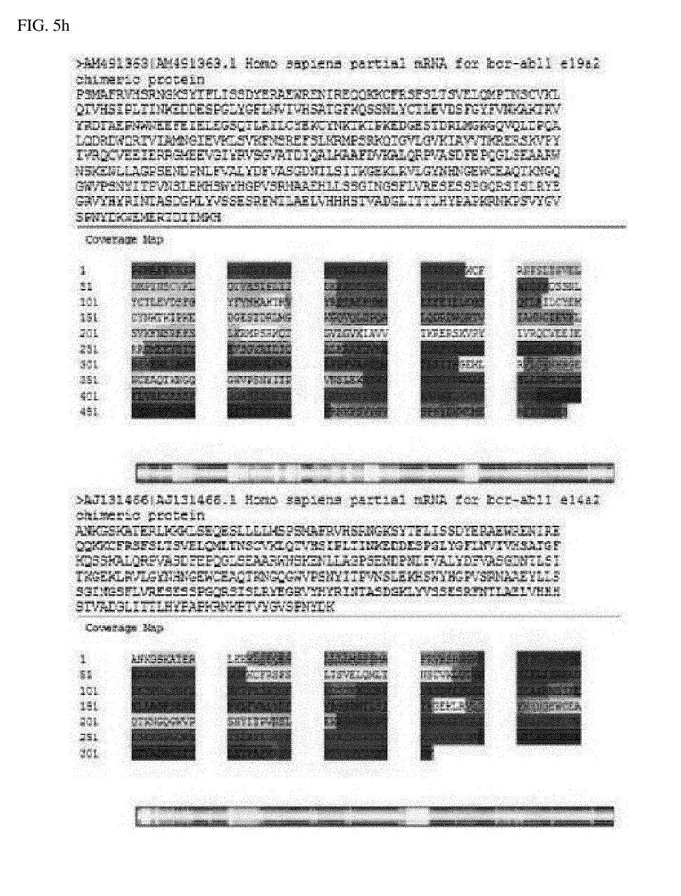

In order to further study molecular and cellular causes of Korean gastric cancer pathogenesis, a relationship between proteins (DEPs) differentially expressed in Korean gastric cancer tissues was studied using a search tool for recurring instances of neighboring genes (STRING) database (www.STRING-db.org). FIG. 2 shows a STRING analysis result. Red dots indicate up-regulated proteins in cancer tissues, and blue dots indicate down-regulated proteins. Lines indicated by colors between the dots indicate various types of relationships. The differentially expressed proteins (DEPs) were divided into six clusters including most of highly related DEPs and five groups having known or predicted similar functions without relationships between the groups.

Changes in Protein Expression Regulating Actin Cytoskeleton and Motor Activity

Cluster I included two major pathways, that is, components for actin cytoskeleton and motor activity regulation. Interestingly, actin-binding proteins such as actinin α-4 (ACTN4), actinin α-1, actinin α-1 skeletal muscle, Moesin (MSN), Vinculin and Transgelin and cross-linkers were consistently down-regulated in gastric cancer. Expression levels of ACTN4 in cancers of pancreas, ovary, lung, and salivary gland and a MSN level in breast cancer were changed. In contrast, levels of actin polymerized regulatory proteins such as an actin-related protein 3 homolog, an IQ motif-containing GTPase activating protein 1 (IQGAP1), an adenylate cyclase-associated protein 1, a GDP dissociation inhibitor 2 and a Rho GTPase activating protein 1 were significantly increased in gastric cancer. The up-regulation of the IQGAP1 among these proteins is known to be associated with carcinogenesis of lung, ovary, gastric, and colon cancers.

Motor activity regulatory factors such as myosin light chain 9 (MYL9, regulatory), myosin light chain 6, alkali, smooth muscle and non-muscle, tropomyosin 1-α (TPM1), tropomyosin 2-β and tropomyosin 3 (TPM3) were down-regulated in gastric cancer. However, myosin heavy chain 9 (MYH9, non-muscle) was up-regulated. Among these proteins, MYL9, TPM1 and MYH9 are known to be associated with the carcinogenesis of various tumors. Other down-regulation factors include intermediate filament factors such as Vimentin and Desmin (FIG. 1I), which are very closely related to hepatocellular carcinoma and colon cancer, respectively.

Main Components of Significantly Up-Regulated Microtubule

Among various tubulins present in humans, six α-tubulins [tubulin α (TUBA)-1A, TUBA-1B, TUBA-1C, TUBA-3E, TUBA-4A and TUBA-8], seven β-tublines [tubulin β (TUBB), TUBB-2A, TUBB-2B, TUBB-3, TUBB-4A, TUBB-4B, TUBB-6 and TUBB-8], and two regulators [Filamin A (FLNA), and chaperone containing TCP1 subunit 6A] were significantly up-regulated in gastric cancer (FIG. 21I). It is reported that the β-tubulin is an established target for anticancer agents, and the FLNA is up-regulated in breast cancer tissues.

Ion Binding Proteins with Change in Expression in Gastric Cancer

Among proteins DEPs with changes in expression, iron- and oxygen-binding proteins such as hemoglobin (HB) subunit-β, HB subunit-η2, HB subunit-δ, HB subunit-ε1, and HB subunit-η1 were significantly down-regulated in gastric cancer. In addition, the binding of albumin to Ca²⁺, Na⁺, and K⁺ was down-regulated in gastric cancer. On the other hand, other iron-binding proteins, transferrin and zinc-containing carbonic anhydrase were significantly up-regulated in gastric cancer (FIG. 2III).

Up-Regulated Glycolytic Metabolism-Related Proteins in Gastric Cancer

Proteins in Cluster IV are involved in various metabolic processes. Among these proteins, glyceraldehyde-3-phosphate dehydrogenase, enolase 1 (ENO1), enolase 2 (ENO2), glucose-6-phosphate isomerase (GPI), pyruvate kinase muscle (PKM), and phosphoglycerate kinase 1 (PGK1) are highly related to glycolysis. These genes are up-regulated and closely related to each other. This cluster also contains down-regulated malate dehydrogenase 1, ATP synthase H⁺ metastatic mitochondrial F1 complex α-subunit, and citrate synthases found in myocardia and up-regulated mitochondria (FIG. 2IV).

Most of molecular chaperone-related proteins were up-regulated in gastric cancer.

Cluster V (FIG. 2V) includes two central molecular chaperones and corresponds to heat shock protein (HSP) 90 kDa cytoplasm-α class A member 1 and HSP90 α-family class b member 1, and strongly reacts with HSP family A member 1 analogue, HSP family A member 2, HSP family A member 5, HSP family A member 6, HSP family A member 8, HSP family A member 9, HSP family D member 1, hypoxia up-regulated 1 and HSP B member 1 (HSPB1). Except for the HSPB1, all heat shock proteins were up-regulated in gastric cancer.

Up-Regulated Proteins with Protein Folding and Trafficking Activities

Cluster VI (FIG. 2VI) includes up-regulated proteins located in the Golgi body (GA) or the endoplasmic reticulum (ER) and regulates protein folding and trafficking. Valosin-containing protein, major histocompatibility complex class 1 (HLA)-B, HLA-C, ribophorin II (RPN2), protein disulfide isomerase family A (PDIA) member-3, PDIA member-6, prolyl 4-hydroxylase subunit-β, calnexin (CANX), calreticulin (CALR), thioredoxin domain containing 5, and glucosidase II α-subunit are significantly up-regulated proteins.

Up-Regulated Proteins Involved in Protein Synthesis

Three proteins involved in protein synthesis, that is, eukaryotic translation elongation factor (EEF)-2, EEF-1A1 and EEF-1A2 were up-regulated and interacted with factors of other clusters. Among these proteins, the EEF1A2 is associated with carcinogenesis of ovarian cancer.

Down-Regulated Proto-Oncogene and Up-Regulated Protease Inhibitors in Gastric Cancer

Proto-oncogenes such as anterior gradient 2, anterior gradient 3, Ras inhibitor-1, SET nuclear proto-oncogene, tryptophanyl-tRNA synthethase, and ubiquitin-like modifier activating enzyme 1 were significantly down-regulated in gastric cancer (FIG. 2B). In addition, it was found that serpin peptidase inhibitor clade (SERPIN)-A member 1 (SPERPINA1) associated with tumor progression in gastric cancer and colon cancer and SERPIN-H member 1 identified as a strong biomarker of early-stage hepatocellular tumor were significantly increased in gastric cancer in the present invention.

Expression-Change Proteins Associated with Energy Metabolism and Cell Structural Factors

Creatine kinase B regulates energy homeostasis in tissues, and is decreased in cervical cancer and down-regulated in gastric cancer. In contrast, an ATPase Na⁺/K⁺-transfer subunit α-1 which maintains energy homeostasis, mitochondrial aldehyde dehydrogenase 2 family which generates carboxylic acid by oxidizing aldehyde, and gastric type lipase F which is an enzyme involved in the digestion of triglycerides in foods were significantly up-regulated in gastric cancer.

Other proteins with multiple domains important for cellular structural organization also exhibited changes in expression. For example, POTE ankyrin domain family members (POTE)-J and POTE-I were down-regulated and up-regulated in gastric cancer, respectively. Lumican, which belongs to a leucine-rich small proteoglycan family regulating collagen fibril organization and involved in prostate cancer, was significantly down-regulated in gastric cancer. Major vault proteins are highly overexpressed in drug-resistant cancers. Caroferrin subunit β-1, junction plakoglobin, lamin A/C, multiple PDZ domain crumbs cell polarity complex component, clathrin heavy chain, and leucine-rich pentatricopeptide repeat-containing were up-regulated.

The fusion protein was identified by label-free proteome analysis.

The present inventors tested whether a peptide binds to a junction of two proteins by a mass spectrometer according to a manual. Only the hnRNPA2B1-FAM96A fusion protein was identified to have the corresponding peptide binding the two proteins.

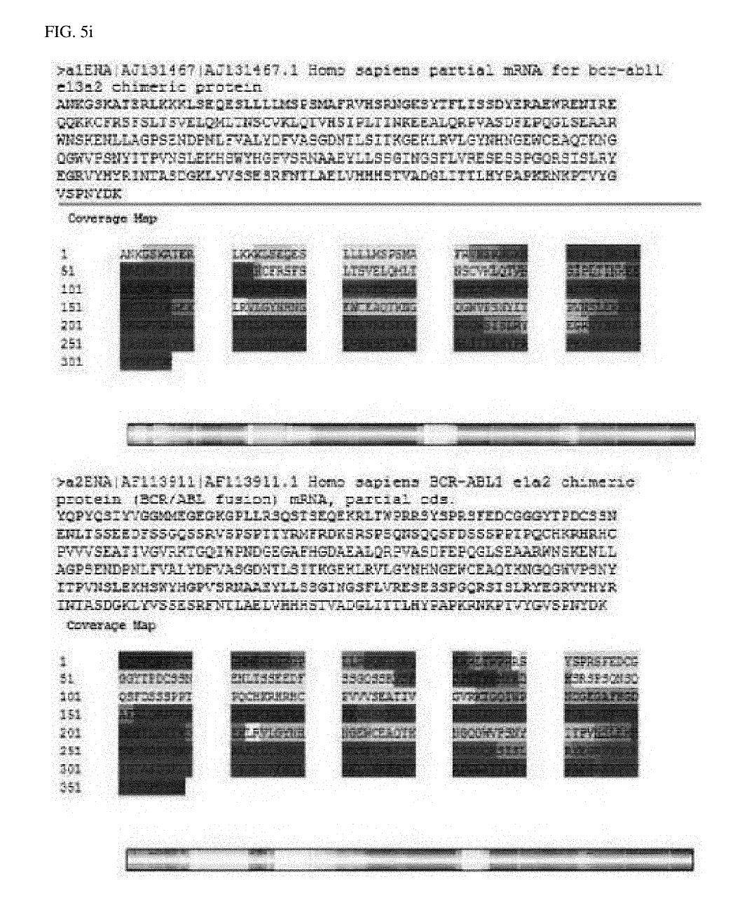

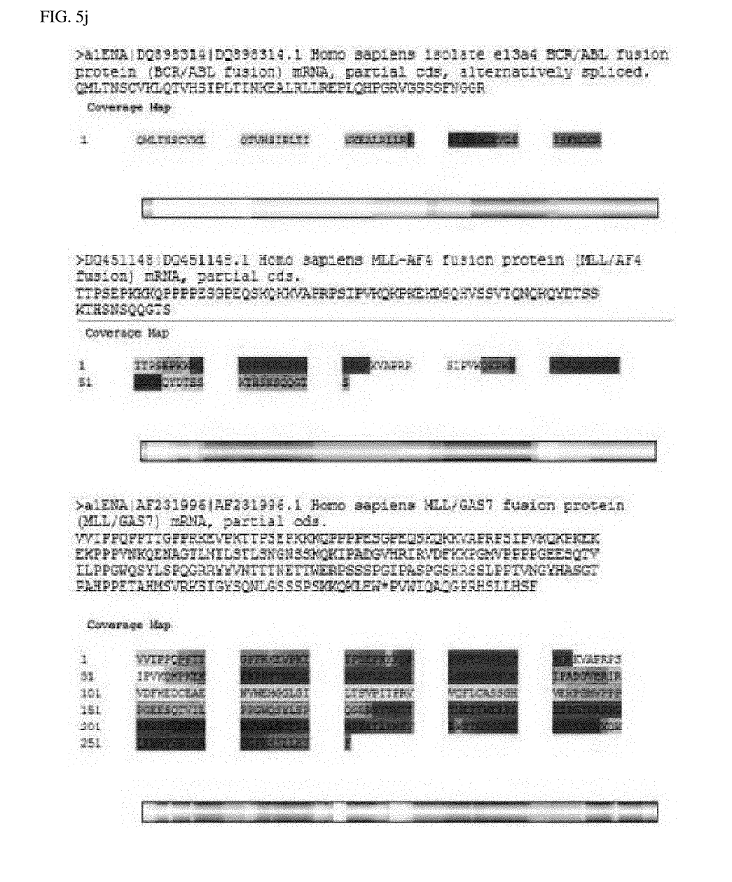

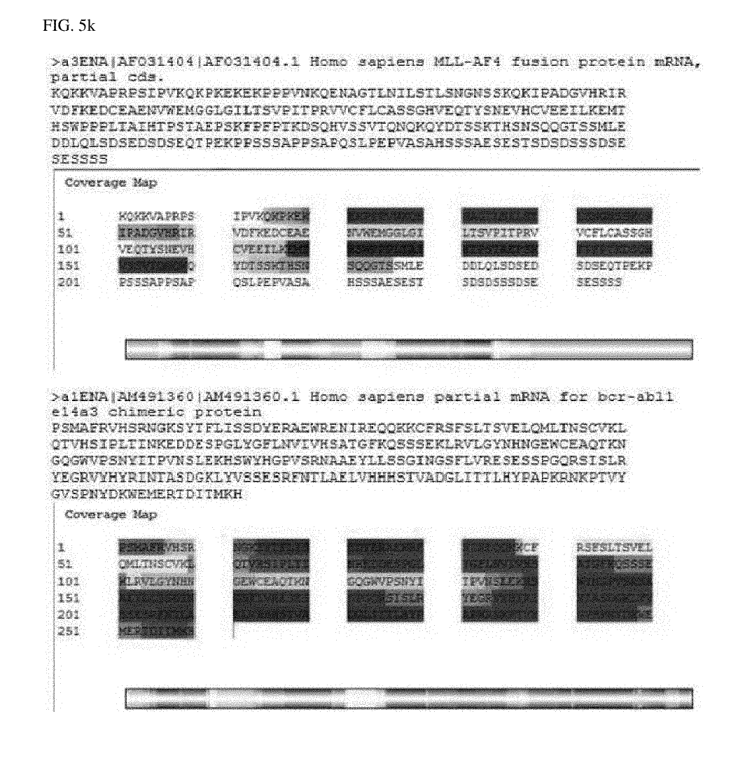

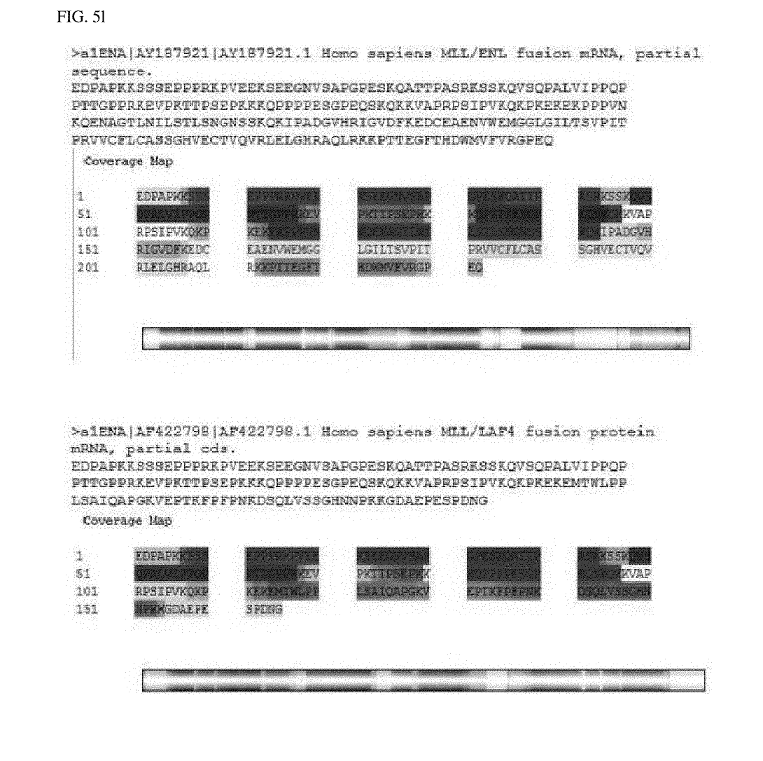

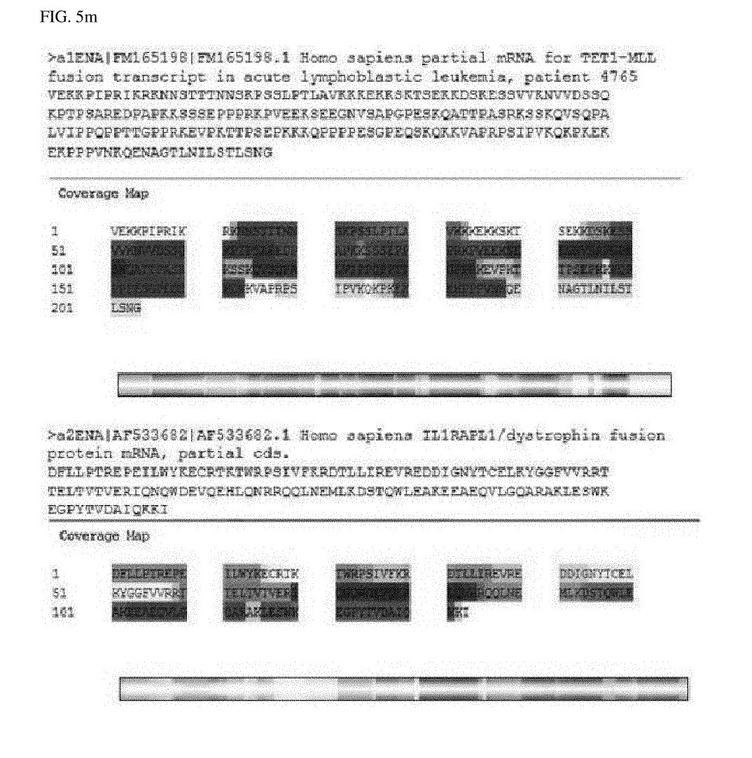

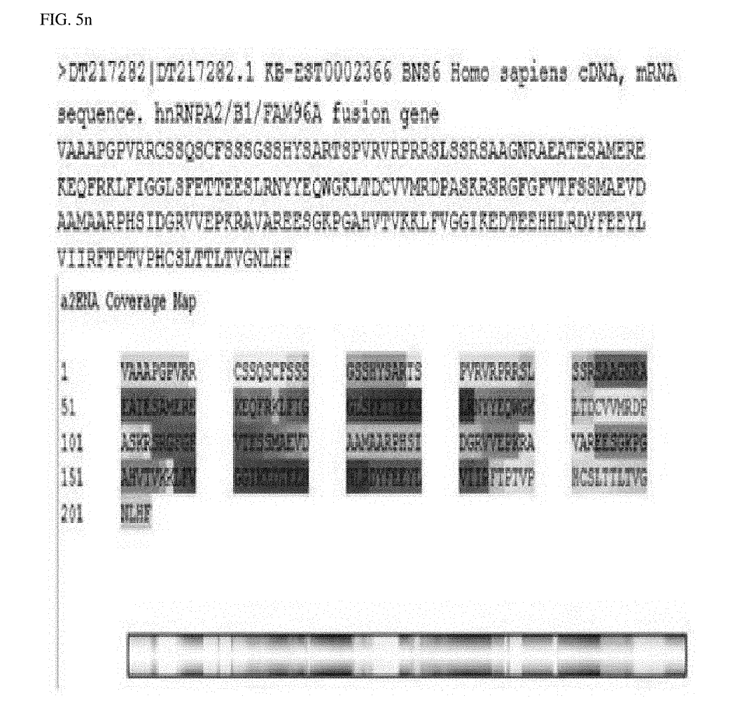

In order to further study a possible role of the hnRNPA2B1-FAM96A fusion protein in the carcinogenesis process, a three-dimensional structure of the hnRNPA2B1-FAM96A fusion protein was predicted by performing a homology modeling. Assuming that the FAM96A is known to form dimers, the three-dimensional modeling of the hnRNPA2B1-FAM96A fusion protein showed that the fusion protein is present as dimers (FIG. 3). The dimerization ability of the fusion protein suggests that the location of the fusion protein may be different from that of the normal protein, thereby changing cellular and molecular processes.

Table 1 shows clinical and pathological data of nine gastric cancer patients.

TABLE 1

| | | | | Tumor | | Lauren |
|---|---|---|---|---|---|---|
| No | Sex | Age | Stage | location | Differentation | classification |
| 1 | F | 82 | IIB | Antrum | Poorly differentiated | Intestinal |
| 2 | M | 57 | IIA | Antrum | Moderately differentiated | Intestinal |
| 3 | M | 56 | IIIB | Antrum | Poorly differentiated | Mixed |
| 4 | M | 79 | IIIB | Antrum | Poorly differentiated | Mixed |
| 5 | M | 78 | IIIB | Antrum | Signet ring cell | Diffuse |
| 6 | M | 78 | IIA | Body | Moderately differentiated | Intestinal |
| 7 | F | 74 | IIIC | Body | Signet ring cell | Diffuse |
| 8 | F | 78 | IIIC | Body | Poorly differentiated | Diffuse |
| 9 | M | 59 | IIIA | Body | Poorly differentiated | Mixed |

1. An hnRNPA2B1-FAM96A fusion protein for diagnosing cancers, wherein a heterogeneous nuclear ribonucleoprotein A2/B1 (hnRNPA2B1) protein or a fragment thereof at an N-terminal and a family with sequence similarity 96 member A (FAM96A) protein or a fragment thereof at a C-terminal are fused.

2. The hnRNPA2B1-FAM96A fusion protein of claim 1, wherein the hnRNPA2B1-FAM96A fusion protein has an amino acid sequence of SEQ ID NO: 1.

3. A hnRNPA2B1-FAM96A fusion gene for diagnosing cancers encoding the fusion protein of claim 1.

4. The hnRNPA2B1-FAM96A fusion gene of claim 3, wherein the fusion gene has a nucleotide sequence of SEQ ID NO: 2.

5. The hnRNPA2B1-FAM96A fusion protein of claim 1, wherein the cancer is any one selected from lung cancer, liver cancer, colon cancer, pancreatic cancer, gastric cancer, breast cancer, ovarian cancer, kidney cancer, thyroid cancer, esophageal cancer, prostate cancer, and brain cancer.

6. A composition for diagnosing cancers of patients to be tested, wherein the composition includes at least one selected from the group consisting of a molecule specifically binding to the fusion protein of claim 1 and a polynucleotide hybridizable with a fusion gene encoding the fusion protein.

7. The composition for diagnosing cancers of claim 6, wherein the molecule specifically binding to the fusion protein is at least one selected from the group consisting of an antibody and an aptamer.

8. The composition for diagnosing cancers of claim 6, wherein the cancer is any one selected from lung cancer, liver cancer, colon cancer, pancreatic cancer, gastric cancer, breast cancer, ovarian cancer, kidney cancer, thyroid cancer, esophageal cancer, prostate cancer, and brain cancer.

9. A method for providing cancer diagnosis information, comprising: detecting the fusion protein of claim 1, a fusion gene encoding the fusion protein, or an mRNA corresponding to the fusion gene, wherein when the fusion protein, the fusion gene, or the mRNA is detected, the patient is determined as a cancer patient.

10. The method for providing cancer diagnosis information of claim 9, wherein the cancer is any one selected from lung cancer, liver cancer, colon cancer, pancreatic cancer, gastric cancer, breast cancer, ovarian cancer, kidney cancer, thyroid cancer, esophageal cancer, prostate cancer, and brain cancer.

11. The method for providing cancer diagnosis information of claim 9, wherein in the detecting, any one method selected from immunochromatography, immunohistochemical staining, ELISA, radioimmunoassay, enzyme immunoassay, fluorescence immunoassay, luminescence immunoassay, western blotting, and FACS is applied.

12. A composition for preventing or treating cancers comprising: at least one selected from the group consisting of an inhibitor of the fusion protein of claim 1 and a polynucleotide molecule inhibitor encoding the fusion protein as an active ingredient.

13. The composition for preventing or treating cancers of claim 12, wherein the inhibitor of the fusion protein includes at least one substance selected from the group consisting of an antibody, an aptamer, a kinase inhibitor, and a signal transduction inhibitor.

14. The composition for preventing or treating cancers of claim 12, wherein the polynucleotide molecule inhibitor includes at least one substance selected from the group consisting of an siRNA, an shRNA, and an aptamer.

15. The composition for preventing or treating cancers of claim 12, wherein the cancer is any one selected from lung cancer, liver cancer, colon cancer, pancreatic cancer, gastric cancer, breast cancer, ovarian cancer, kidney cancer, thyroid cancer, esophageal cancer, prostate cancer, and brain cancer.


SEQUENCE LISTING (2 sequences)

SEQ ID NO: 1 - protein, 150 aa, Artificial Sequence
  synthesized
MEREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYEYLVIIRFTPTVPHCSLTTLTVGNLHF

SEQ ID NO: 2 - DNA, 452 bp, Artificial Sequence
  synthesized
atggagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatgaatatctggttattatcaggttcacgccaacagtacctcattgctctttgacgactcttactgttggaaatctacatttctg